(19) United States
(12) Patent Application Publication
(10) Pub. No.: US 2026/0014320 A1
(43) Pub. Date: Jan. 15, 2026

(54) A sub-assembly of a medicament delivery device

(71) Applicant: SHL Medical AG, Zug (CH)

(72) Inventors: Ming-Ting Yin, Taoyuan City (TW); Pei Yu Chao, Taoyuan City (TW); Yun Hsuan Chang, Taoyuan City (TW)

(21) Appl. No.: 18/880,952

(22) PCT Filed: Jun. 21, 2023
(86) PCT No.: PCT/EP2023/066771

§ 371 Date: Jan. 3, 2025

(30) Foreign Application Priority Data
Jul. 5, 2022 (EP) 22182957.5

(51) Int. Cl. A61M 5/24; A61M 5/31; A61M 5/34

(52) U.S. Cl. CPC A61M 5/24; A61M 5/3129; A61M 5/344; A61M 2005/2407; A61M 2005/2477

(57) ABSTRACT

The present disclosure provides a sub-assembly of a medicament delivery device, the sub-assembly including a housing extending along a longitudinal axis between a distal end of the housing and a proximal end of the housing. The housing is configured to accommodate a first medicament container with a first dimension and a first volume. The sub-assembly further includes an adapter comprising an adapter body extending in the direction of the longitudinal axis from a distal end of the adapter to a proximal end of the adapter. The adapter body is arranged within the housing, and the adapter body is configured to accommodate a second medicament container dimensioned differently from the first dimension and capable of containing no more than the first volume of a medicament. The adapter body is configured to operably retain the second medicament container within the housing so that the second medicament container is radially immovable within the housing.

### CROSS-REFERENCE TO RELATED APPLICATIONS

The present application is a U.S. National Phase application pursuant to 35 U.S.C. § 371 of International Application No. PCT/EP2023/0665771 filed Jun. 21, 2023, which claims priority to EP patent application Ser. No. 22/182,957.5 filed Jul. 5, 2022. The entire disclosure contents of these applications are herewith incorporated by reference into the present application.

### TECHNICAL FIELD

The present disclosure generally relates to a sub-assembly of a medicament delivery device, and particularly to a sub-assembly of a medicament delivery device with an adapter and a delivery member holder assembly.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments such as a growth hormone.

Mass production of medicament delivery devices can reduce the cost of each medicament delivery device and thus make the medicament delivery device affordable for more patients. However, the differing properties of different medicaments makes that difficult.

Therefore, a medicament delivery device with more standardized components is desirable.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device. The sub-assembly comprising a housing extending along a longitudinal axis between a distal end of the housing and a proximal end of the housing. The housing is configured to accommodate a first medicament container with a first dimension and a first volume. An adapter comprising an adapter body extending in the direction of the longitudinal axis from a distal end of the adapter to a proximal end of the adapter. The adapter body is arranged within the housing. The adapter body is configured to accommodate a second medicament container dimensioned differently from the first dimension and capable of containing no more than the first volume of a medicament. The adapter body is configured to operably retain the second medicament container within the housing so that the second medicament container is radially immovable within the housing. A delivery member holder assembly is arranged at least partially within the housing. The delivery member holder assembly comprises a retainer and a delivery member hub configured to be attached to a medicament delivery member. The retainer comprises a body attached to the housing and a central cavity extending through the body in the direction of the longitudinal axis. The central cavity is configured to be lined up with a proximal end of the second medicament container when the second medicament container is accommodated in the adapter body. The delivery member hub is movable within the central cavity from a proximal position where the delivery member hub is configured to keep the medicament delivery member spaced apart from the second medicament container to a distal position where the delivery member hub is configured to enable the medicament delivery member to establish fluid communication with the second medicament container.

Preferably, according to another embodiment, the retainer comprises a delivery member fastener extending from a surface of the central cavity of the body of the retainer. The delivery member hub comprises a counter delivery member fastener extending from an outer surface of the delivery member hub. The counter delivery member fastener is engaged with the delivery member fastener. One of the delivery member fastener of the retainer and the counter delivery member fastener of the delivery member hub is a screw thread; and the other one of the delivery member fastener of the retainer and the counter delivery member fastener of the delivery member hub is a thread follower.

Preferably, according to another embodiment, the thread follower is a screw thread structure, a protruding pin, or a helical protrusion.

Preferably, according to another embodiment, the body of the retainer comprises a distally directed surface configured to be in contact with a shoulder section of the second medicament container.

Preferably, according to another embodiment, the body of the retainer comprises a distally extending section. The body of the retainer comprises a housing fastener configured to be connected to the housing so that the retainer is attached to the housing. The fastener of the retainer is a part of the distally extending section of the body of the retainer.

Preferably, according to another embodiment, the distally extending section of the body of the retainer is a distally extending arm.

Preferably, according to another embodiment, the adapter body is at least partially located between the distally extending section of the body of the retainer and the longitudinal axis in a direction transverse to the longitudinal axis. The housing fastener of the retainer is engaged with a part of the housing so that the retainer is directly attached to the housing.

Alternatively, according to another embodiment, the adapter body is at least partially located between the distally extending section of the body of the retainer and the housing in a direction transverse to the longitudinal axis. The adapter body is engaged with the housing. The housing fastener of the retainer is engaged with the adapter body so that the retainer is indirectly attached to the housing via the adapter body.

Preferably, according to another embodiment, the adapter body is attached to the housing via a form-fit, a snap-fit, a press-fit engagement, or a combination thereof.

Preferably, according to another embodiment, the housing fastener comprises a snap-fit ledge extending in a direction transverse to the longitudinal axis.

Preferably, according to another embodiment, the adapter comprises an extending section extending from the distal end of the adapter body in a direction transverse to the longitudinal axis and towards the longitudinal axis. The extending section comprises a proximally directed surface configured to engage with a distal end of the second medicament container so that the second medicament container is axially immovable within the housing.

Preferably, according to another embodiment, the adapter body comprises a sleeve configured to surround the second medicament container.

Preferably, according to another embodiment, the sleeve is formed by a distal sleeve and a proximal sleeve attached to the distal sleeve; or the sleeve is formed by two halves that are each a semi-cylindrical sleeve body.

Another aspect of the invention provides a medicament delivery device comprising the sub-assembly.

Another aspect of the invention provides a cassette of a medicament delivery device comprising the sub-assembly.

Another aspect of the invention provides a medicament delivery device comprising the cassette.

Preferably, according to another embodiment, the medicament delivery device comprises a reusable drive unit configured to be releasably attached to the cassette unit.

Preferably, according to another embodiment, the medicament delivery device comprises a multiple-chamber medicament container arranged within the carrier.

Preferably, according to another embodiment, the medicament delivery device is an auto-injector or a manual insulin pen.

Preferably, according to another embodiment, the medicament delivery device is an auto-injection with only one spring.

Preferably, according to another embodiment, the first and/or the second medicament container is a medicament container without a medicament delivery member.

Preferably, according to another embodiment, the first and/or the second medicament container is a glass cartridge, a plastic cartridge, or a collapsible bag.

Preferably, according to another embodiment, the medicament delivery member is an injection needle, an infusion catheter, or a spray nozzle.

Another aspect of the invention provides a method of assembling a sub-assembly of a cassette of a medicament delivery device. The method comprising the steps of: providing a group of housings, wherein each housing in the group of housings is identical to one another; providing a group of medicament containers, wherein the group of medicament container comprises a first medicament container (M1) with a first dimension and a first volume and a second medicament container (M2) with a second dimension; and wherein second dimension is different from the first dimension and capable of containing no more than the first volume of a medicament; providing a group of delivery member holder assemblies, wherein each of the delivery member holder assembly in the group of delivery member holder assemblies comprises a retainer and a delivery member hub configured to be attached to a medicament delivery member; wherein the retainer comprises a body and a central cavity extending through the body in the direction of the longitudinal axis; wherein the delivery member hub is movable within the central cavity; providing an adapter; selecting a medicament container from the group of medicament containers; selecting a delivery member holder assembly from the group of the delivery member holder assemblies; inserting the selected medicament container into the housing when the first medicament container is selected; or connecting the selected medicament container to the adapter when the second medicament container is selected, and inserting the selected medicament container with the adapter into the housing; and attaching the selected delivery member holder assembly to the housing so that the central cavity of the retainer is lined up with the selected medicament container.

Preferably, according to another embodiment, the step of providing a group of delivery member holder assemblies, comprising the steps of: providing the group of delivery member holder assemblies, wherein the group of delivery member holder assemblies comprises a first retainer configured to snugly fit to the first medicament container and a second retainer configured to snugly fit to the second medicament container; and when the second medicament container is selected, attaching the adapter to an outer surface second retainer when the second retainer is selected, or attaching the adapter to an inner surface of the housing when the second retainer is selected; thereby connecting the second medicament container indirectly to the adapter via the second retainer.

Preferably, according to another embodiment, the step of providing a group of delivery member holder assemblies, comprising the steps of: providing the group of delivery member holder assemblies, each delivery member holder assembly of the group of delivery member holder assemblies is identical to one another; and when the second medicament container is selected, attaching the adapter to the second medicament container, thereby positioning the adapter and the second medicament container at least partially within the retainer of the delivery member holder assembly when attaching the selected delivery member holder assembly to the housing.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a cross-section view of a sub-assembly of the invention without a housing of the sub-assembly.

FIG. 2 schematically shows a cross-section view of the sub-assembly of FIG. 1 within the housing of the sub-assembly.

FIGS. 3A-C schematically show perspective views of an adapter of the sub-assembly of FIG. 1 in different embodiments.

FIG. 4 schematically shows a perspective view of the sub-assembly of the invention in another embodiment.

FIG. 5 schematically shows a perspective view of the sub-assembly of FIG. 4.

FIG. 6 schematically shows a cross-section view of the sub-assembly of FIG. 4.

### DETAILED DESCRIPTION

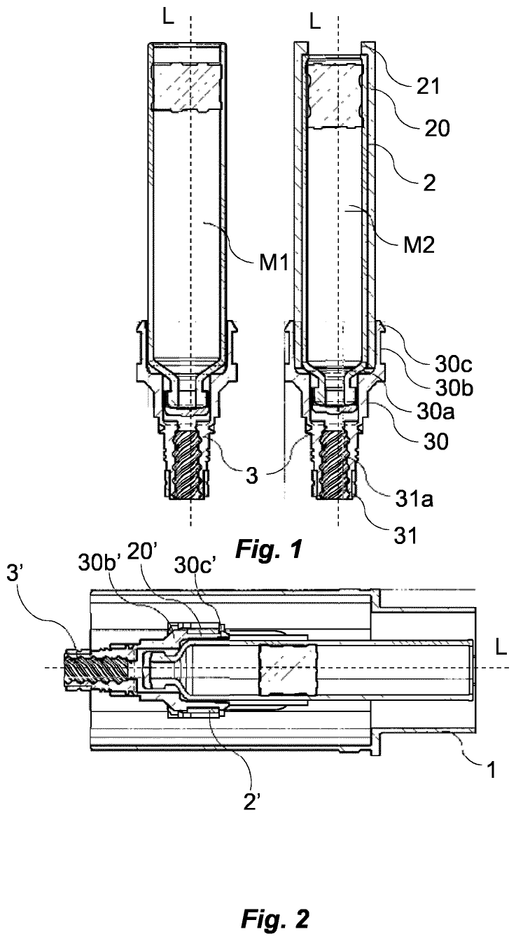

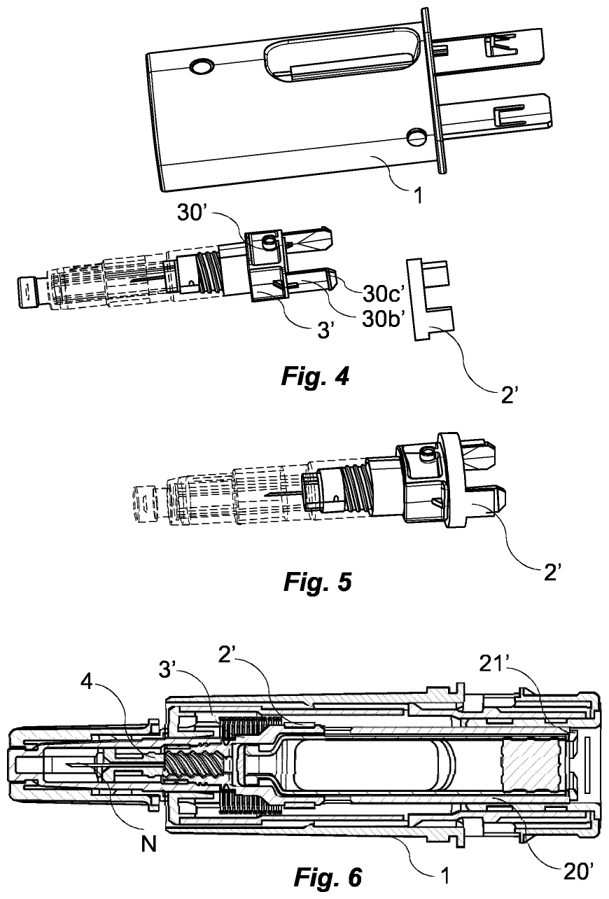

FIGS. 1-6 illustrate a sub-assembly of a medicament delivery device. The sub-assembly comprises a housing 1, an adapter 2; 2′, and a delivery member assembly 3, 4. The housing 1 extends along a longitudinal axis L between a distal end of the housing 1 and a proximal end of the housing 1. The housing 1 is configured to accommodate a first medicament container M1 with a first dimension and a first volume. In one example, the housing is configured to match the shape of the first medicament container M1. For example, the housing is generally cylindrical as the medicament containers are commonly cylindrical.

The adapter 2; 2′ comprises an adapter body 20; 20′; 20″; 20′″; 20″″ extending in the direction of the longitudinal axis L from a distal end of the adapter 2; 2′ to a proximal end of the adapter 2; 2′. The adapter body 20; 20′; 20″; 20′″; 20″″ is arranged within the housing 1.

The adapter body 20; 20′; 20″; 20′″; 20″″ is configured to accommodate a second medicament container M2 dimensioned differently from the first dimension, namely, the second medicament container M2 has a different measured length and/or width and/or heigh and/or diameter with the first medicament container M1. For example, the second medicament container has a shorter length than the first medicament container when measured along the longitudinal axis L.

Alternatively, when the first and the second medicament container are both cylindrical, the second medicament container has a smaller outer diameter than the first medicament container. Alternatively, when the first and the second medicament container are both cylindrical, the second medicament container has a smaller outer diameter than the first medicament container, and the second medicament container has a shorter length than the first medicament container when measured along the longitudinal axis L.

The second medicament container M2 is capable of containing no more than the first volume of a medicament. For example, the first medicament container M1 is a 5 mL cartridge, and the second medicament container M2 is a 3 mL cartridge.

The adapter body 20; 20′; 20″; 20″′; 20″″ is configured to operably retain the second medicament container M2 within the housing 1 so that the second medicament container M2 is radially immovable within the housing 1. The adapter body 20; 20′; 20″; 20″′; 20″″ can be attached to the second medicament container M2 directly; or can be attached to another component that is configured to be attached to the second medicament container M2 directly. The connection between the adapter 2; 2′ and the second medicament container M2; and the connection between the adapter 2; 2′ and the housing 1 will be explained in detail later.

The sub-assembly is configured to be used with a medicament container without an integral medicament delivery member, e.g., a needle or a nozzle. For example, the first medicament container and the second medicament container are cartridges or are collapsible bags. Thus, the sub-assembly comprises the delivery member holder assembly 3; 3′, 4 arranged at least partially within the housing 1. The delivery member holder assembly 3; 3′, 4 comprises a retainer 3; 3′ and a delivery member hub 4. The delivery member hub 4 is configured to be attached to a medicament delivery member, e.g., by being glued to the medicament delivery member.

The retainer 3; 3′ comprises a body 30; 30′ attached to the housing and a central cavity 31 extending through the body 30; 30′ in the direction of the longitudinal axis L. The central cavity 31 is configured to be lined up with a proximal end of the second medicament container M2 when the second medicament container M2 is accommodated in the adapter body 20; 20′; 20″; 20″′; 20″″. The delivery member hub 4 is movable within the central cavity 31 from a proximal position where the delivery member hub 4 is configured to keep the medicament delivery member N spaced apart from the second medicament container M2 to a distal position where the delivery member hub 4 is configured to enable the medicament delivery member N to establish fluid communication with the second medicament container M2.

In one example, the retainer 3 comprises a delivery member fastener 31a extending from a surface of the central cavity 31 of the body 30; 30′ of the retainer. The delivery member hub 4 comprises a counter delivery member fastener (not shown) extending from an outer surface of the delivery member hub 4. The counter delivery member fastener is configured to be engaged with the delivery member fastener 31a. In one example, one of the delivery member fastener 31a of the retainer 3; 3′ and the counter delivery member fastener of the delivery member hub 4 is a screw thread and the other one of the delivery member fastener 31a of the retainer 3 and the counter delivery member fastener of the delivery member hub 4 is a thread follower. For example, as shown in FIGS. 1-2, the delivery member fastener 31a of the retainer 3; 3′ is a screw thread. In this example, the counter delivery member fastener of the delivery member hub 4 is a thread follower, e.g., a protruding pin positioned within the screw thread or a helical protrusion that is configured to be moved along the screw thread or another screw thread matching with the screw thread of the delivery member fastener 31a.

In one example, the body 30 of the retainer 3 comprises a distally directed surface 30a configured to be in contact with a shoulder section of the second medicament container M2, as shown in FIG. 1.

In one example, as shown in FIGS. 1-2, the body 30; 30′ of the retainer 3; 3′ comprises a distally extending section 30b; 30b′. The body 30; 30′ of the retainer 3; 3′ comprises a housing fastener 30c; 30c′ configured to be connected to the housing 1 so that the retainer 3; 3′ is attached to the housing 1. In this example, the housing fastener 30c; 30c′ of the retainer 3; 3′ is a part of the distally extending section 30b; 30b′ of the body 30; 30′ of the retainer 3; 3′. For example, as shown in FIG. 1, the housing fastener 30c of the retainer 3 is a ledge extending in a direction transverse to the longitudinal axis L. In this example, the retainer 3 is attached to the housing 1 directly via a snap-fit connection between the housing fastener 30c and a counter retainer fastener of the housing 1. In this example, the distally extending section 30b of the body 30 of the retainer 3 is a distally extending arm 30b. Alternatively, the housing fastener is defined by a side wall of the distally extending section of the body of the retainer. In this example, the side wall of the distally extending section of the body of the retainer comprises a rubber material part configured to be attached to an inner side wall of the housing via a press-fit connection.

In one example, the adapter 2; 2′ is configured to interact with the retainer 3; 3′. In one example, the adapter body 20 is at least partially located between the distally extending section of the body 30b of the retainer 3 and the longitudinal axis L in the direction transverse to the longitudinal axis L. In this example, the housing fastener 30c of the retainer 3 is engaged with a part of the housing 1 so that the retainer 3 is directly attached to the housing 1, as mentioned above. In this example, the retainer 3 and the housing 1 are identical for both the first medicament container M1 and the second medicament container M2. As the shape of the delivery member hub 4 is mainly relevant to the shape of the retainer 3, in this example, the delivery member holder assembly 3, 4 can be identical for both the first medicament container M1 and the second medicament container M2. In this example, the adapter 2 is configured to retain the second medicament container M2 indirectly to the housing via the retainer 3. In this example, the adapter 2 is configured to fill up the radial gap between the second medicament container M2 and the body 30 of the retainer 3. The adapter 2 can be a hard-shell sleeve or can be made of resilient material, therefore, the medicament container M2 is immovable relative to the body 30 of the retainer 3 in the radial direction. As the retainer is attached to the housing 1, the second medicament container is also attached to the housing 1 via the retainer 3. It should be noted that as the housing 1 is configured to accommodate the first medicament container M1, when the housing 1 accommodate the first medicament container M1, it is not necessary to use the adapter 2.

Alternatively, in another example, the adapter body 20′ is at least partially located between the distally extending section 30b′ of the body 30′ of the retainer 3′ and the housing 1 in the direction transverse to the longitudinal axis L, as shown in FIG. 2. In this example, the adapter body 20′ is engaged with the housing 1 directly. In this example, the housing fastener 30c′ of the retainer 3′ is engaged with the adapter body 20′ so that the retainer 3′ is indirectly attached to the housing 1 via the adapter body 20′. In this example, the adapter body 20′ is attached to the housing 1 via a form-fit, a snap-fit, a press-fit engagement, or a combination thereof. For example, the adapter body 20′ is made of resilient material and is configured to be attached to the housing via a press-fit engagement. In this example, the retainer for the first medicament container M1 is different from the retainer 3′ for the second medicament container M2. Therefore, the radial gap between the second medicament container 2 and the body 30′ of the retainer 3′ can be minimized. In this example, the adapter 2′ is not necessary to contact the second medicament container M2. In this example, the adapter 2′ retains the second medicament container M2 indirectly via retaining the retain 3′ to the housing 1. In a preferred example, the housing fastener 30c′ of the body 30′ of the retainer 3′ comprises a snap-fit ledge extending in the direction transverse to the longitudinal axis L.

It should be noted that medicament delivery devices are commonly arranged with a power source with a plunger rod next to a distal end of a contained medicament container. Therefore, it is not necessary that the adapter prevents the second medicament container M2 from moving axially, as the power source with the plunger rod will limit the axial movement of the second medicament container M2 once the sub-assembly as disclosed is connected to the power source with the plunger rod of the medicament delivery device.

Alternatively, the adapter 2; 2′ can provide axial support to the second medicament container M2. In this example, the adapter 2; 2′ comprises an extending section 21; 21′ extending from the distal end of the adapter body 20; 20′ in the direction transverse to the longitudinal axis L and towards the longitudinal axis L, as shown in FIG. 1 and FIG. 6. In this example, the extending section 21; 21′ comprises a proximally directed surface configured to engage with a distal end of the second medicament container M so that the second medicament container is axially immovable within the housing 1.

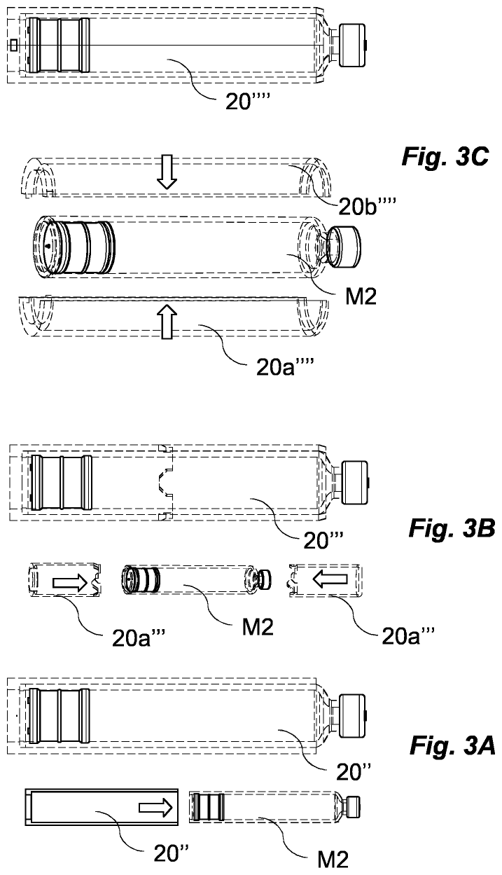

In one example, the adapter body 20″ comprises a sleeve 20″ configured to surround the second medicament container M, as shown in FIG. 3A. In another example, the sleeve 20″′ is formed by a distal sleeve 20a″′ and a proximal sleeve 20b″′ attached to the distal sleeve 20a″′, as shown in FIG. 3B. Alternatively, the sleeve 20″″ is formed by two halves 20a″″, 20b″″ that are each a semi-cylindrical sleeve body, as shown in FIG. 3C.

The sub-assembly can be used in a medicament delivery device that is configured to be fully assembled in a manufacturing site and delivered to an end-user, e.g., a patient. In this example, the medicament delivery device can be assembled either by assembling the first medicament container M1 directly into the housing 1 or by assembling the second medicament container M2 with the adapter 2; 2′ into the housing 1 in the production line.

Alternatively, the sub-assembly can be used in a cassette unit for a medicament delivery device. In this example, the medicament delivery device comprises a reusable drive unit configured to be releasably attached to the cassette unit. In this example, the cassette unit can be fully assembled at a manufacturing site or can be fully assembled later. For example, the cassette unit can be fully assembled in a pharmacy or by a doctor. In this example, the pharmacist can either assemble the first medicament container M1 directly into the housing 1 or assemble the second medicament container M2 with the adapter 2; 2′ into the housing 1.

When the sub-assembly is used in a cassette of a medicament delivery device or a medicament delivery device without a cassette, e.g., a preassembled disposable medicament delivery device, the sub-assembly can be assembled with the following method.

Firstly, the method comprises the steps of providing a group of housings, a group of medicament containers, a group of delivery member holder assemblies, and the adapter 2; 2′.

Each housing in the group of housings is identical to one another. The group of medicament container comprises a first medicament container M1 with a first dimension and a first volume and a second medicament container M2 with a second dimension; wherein the second dimension is different from the first dimension and capable of containing no more than the first volume of a medicament. In the group of delivery member holder assemblies, each of the delivery member holder assemblies in the group of delivery member holder assemblies comprises a retainer and a delivery member hub configured to be attached to a medicament delivery member. The retainer comprises a body and a central cavity extending through the body in the direction of the longitudinal axis L. The delivery member hub is movable within the central cavity.

Secondly, the method comprises the steps of selecting a medicament container from the group of medicament containers and a delivery member holder assembly from the group of delivery member holder assemblies.

Thirdly, the method comprises the following steps in the following order:
inserting the selected medicament container into the housing after the first medicament container is selected; or connecting the selected medicament container to the adapter after the second medicament container is selected and inserting the selected medicament container with the adapter into the housing; and
attaching the selected delivery member holder assembly to the housing so that the central cavity of the retainer is lined up with the selected medicament container.

Furthermore, in another example, the group of delivery member holder assemblies comprise a first retainer configured to snugly fit to the first medicament container and a second retainer configured to snugly fit to the second medicament container. In this example, When the second medicament container is selected, the method comprises the steps of
attaching the adapter to an outer surface second retainer, or
attaching the adapter to an inner surface of the housing; thereby connecting the second medicament container indirectly to the adapter via the second retainer.

Alternatively, in another example, the delivery member holder assemblies of the group of delivery member holder assemblies are identical to one another. In this example, when the second medicament container is selected, the method comprises the step of
attaching the adapter to the second medicament container, thereby positioning the adapter and the second medicament container at least partially within the retainer of the delivery member holder assembly when attaching the selected delivery member holder assembly to the housing.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A sub-assembly of a medicament delivery device, the sub-assembly comprising:
   a housing (1) extending along a longitudinal axis (L) between a distal end of the housing (1) and a proximal end of the housing (1); wherein the housing (1) is configured to accommodate a first medicament container (M1) with a first dimension and a first volume;
   an adapter (2; 2′) comprising an adapter body (20; 20′; 20″; 20″′; 20″″) extending in the direction of the longitudinal axis (L) from a distal end of the adapter (2; 2′) to a proximal end of the adapter (2; 2′); wherein the adapter body (20; 20′; 20″; 20″′; 20″″) is arranged within the housing (1); wherein the adapter body (20; 20′; 20″; 20′″; 20″″) is configured to accommodate a second medicament container (M2) dimensioned differently from the first dimension and capable of containing no more than the first volume of a medicament; wherein the adapter body (20; 20′; 20″; 20′″; 20″″) is configured to operably retain the second medicament container (M2) within the housing (1) so that the second medicament container (M2) is radially immovable within the housing (1);
   a delivery member holder assembly (3; 3′, 4) arranged at least partially within the housing (1); wherein the delivery member holder assembly (3; 3′, 4) comprises a retainer (3; 3′) and a delivery member hub (4) configured to be attached to a medicament delivery member (N); wherein the retainer (3; 3′) comprises a body (30; 30′) attached to the housing and a central cavity (31) extending through the body (30; 30′) in the direction of the longitudinal axis (L); wherein the central cavity (31) is configured to be lined up with a proximal end of the second medicament container (M2) when the second medicament container (M2) is accommodated in the adapter body (20; 20′; 20″; 20″′; 20″″); and wherein the delivery member hub (4) is movable within the central cavity (31) from a proximal position where the delivery member hub (4) is configured to keep the medicament delivery member (N) spaced apart from the second medicament container (M2) to a distal position where the delivery member hub (4) is configured to enable the medicament delivery member (N) to establish fluid communication with the second medicament container (M2).
2. The sub-assembly according to clause 1, wherein the retainer comprises a delivery member fastener extending from a surface of the central cavity of the body of the retainer; wherein the delivery member hub comprises a counter delivery member fastener extending from an outer surface of the delivery member hub; wherein the counter delivery member fastener is engaged with the delivery member fastener; wherein one of the delivery member fastener of the retainer and the counter delivery member fastener of the delivery member hub is a screw thread; and wherein the other one of the delivery member fastener of the retainer and the counter delivery member fastener of the delivery member hub is a thread follower.
3. The sub-assembly according to clause 2, wherein the thread follower is a screw thread structure, a protruding pin, or a helical protrusion.
4. The sub-assembly according to any one of the preceding clauses, wherein the body of the retainer comprises a distally directed surface configured to be in contact with a shoulder section of the second medicament container.
5. The sub-assembly according to any one of the preceding clauses, wherein the body of the retainer comprises a distally extending section; wherein the body of the retainer comprises a housing fastener configured to connect to the housing so that the retainer is attached to the housing; and wherein the housing fastener of the retainer is a part of the distally extending section of the body of the retainer.
6. The sub-assembly according to clause 5, wherein the distally extending section of the body of the retainer is a distally extending arm.
7. The sub-assembly according to clause 5 or 6, wherein the adapter body is at least partially located between the distally extending section of the body of the retainer and the longitudinal axis in a direction transverse to the longitudinal axis; and wherein the housing fastener of the retainer is engaged with a part of the housing so that the retainer is directly attached to the housing.
8. The sub-assembly according to clause 5 or 6, wherein the adapter body is at least partially located between the distally extending section of the body of the retainer and the housing in a direction transverse to the longitudinal axis; wherein the adapter body is engaged with the housing; and wherein the housing fastener of the retainer is engaged with the adapter body so that the retainer is indirectly attached to the housing via the adapter body.
9. The sub-assembly according to clause 8, wherein the adapter body is attached to the housing via a form-fit, a snap-fit, a press-fit engagement, or a combination thereof.
10. The sub-assembly according to any of clauses 5-9, wherein the housing fastener comprises a snap-fit ledge extending in a direction transverse to the longitudinal axis.
11. The sub-assembly according to any one of the preceding clauses, wherein the adapter comprises an extending section extending from the distal end of the adapter body in a direction transverse to the longitudinal axis and towards the longitudinal axis; and wherein the extending section comprises a proximally directed surface configured to engage with a distal end of the second medicament container so that the second medicament container is axially immovable within the housing.
12. The sub-assembly according to any one of the preceding clauses, wherein the adapter body comprises a sleeve configured to surround the second medicament container.
13. The sub-assembly according to clause 12, wherein the sleeve is formed by a distal sleeve and a proximal sleeve attached to the distal sleeve; or wherein the sleeve is formed by two halves that are each a semi-cylindrical sleeve body.
14. A medicament delivery device comprising a sub-assembly according to any of the preceding clauses.
15. A cassette unit of a medicament delivery device comprising a sub-assembly according to any of the preceding clauses.
16. A medicament delivery device comprising the cassette unit according to clause 15, wherein the medicament delivery device comprises a reusable drive unit configured to be releasably attached to the cassette unit.
17. The medicament delivery device according to clause 14 or 16, wherein the medicament delivery device comprises the second medicament container; and wherein the second medicament container is a cartridge or a collapsible bag.
18. A method of assembling a sub-assembly of a cassette of a medicament delivery device, the method comprising the steps of:
   providing a group of housings, wherein the housings in the group of housings are identical;
   providing a group of medicament containers, wherein the group of medicament containers comprises a first medicament container (M1) with a first dimension and a first volume and a second medicament container (M2) with a second dimension; and wherein the second dimension is different from the first dimension and capable of containing no more than the first volume of a medicament;
   providing a group of delivery member holder assemblies, wherein each of the delivery member holder assemblies in the group of delivery member holder assemblies comprises a retainer and a delivery member hub configured to be attached to a medicament delivery member; wherein the retainer comprises a body and a central cavity extending through the body in the direction of the longitudinal axis; wherein the delivery member hub is movable within the central cavity;
   providing an adapter;
   selecting a medicament container from the group of medicament containers;
   selecting a delivery member holder assembly from the group of the delivery member holder assemblies;
   inserting the selected medicament container into the housing when the first medicament container is selected; or connecting the selected medicament container to the adapter when the second medicament container is selected, and inserting the selected medicament container with the adapter into the housing; and
   attaching the selected delivery member holder assembly to the housing so that the central cavity of the retainer is lined up with the selected medicament container.
19. The method according to clause 18, wherein the step of providing a group of delivery member holder assemblies comprises the steps of:
   providing the group of delivery member holder assemblies, wherein the group of delivery member holder assemblies comprises a first retainer configured to snugly fit to the first medicament container (M1) and a second retainer configured to snugly fit to the second medicament container (M2); and
   when the second medicament container is selected, attaching the adapter to an outer surface of the second retainer when the second retainer is selected, or attaching the adapter to an inner surface of the housing when the second retainer is selected; thereby connecting the second medicament container (M2) indirectly to the adapter via the second retainer.
20. The method according to clause 18, wherein the step of providing a group of delivery member holder assemblies comprises the steps of:
   providing the group of delivery member holder assemblies, wherein the delivery member holder assemblies of the group of delivery member holder assemblies are identical; and when the second medicament container (M2) is selected, attaching the adapter to the second medicament container (M2), thereby positioning the adapter and the second medicament container (M2) at least partially within the retainer of the delivery member holder assembly when attaching the selected delivery member holder assembly to the housing.

1-15. (canceled)

16. A sub-assembly of a medicament delivery device, the sub-assembly comprising:
a housing extending along a longitudinal axis between a distal end of the housing and a proximal end of the housing, wherein the housing is configured to accommodate a first medicament container with a first dimension and a first volume;
an adapter comprising an adapter body extending in the direction of the longitudinal axis from a distal end of the adapter to a proximal end of the adapter, wherein the adapter body is arranged within the housing, wherein the adapter body is configured to accommodate a second medicament container dimensioned differently from the first dimension and capable of containing no more than the first volume of a medicament, wherein the adapter body is configured to operably retain the second medicament container within the housing so that the second medicament container is radially immovable within the housing; and
a delivery member holder assembly arranged at least partially within the housing, wherein the delivery member holder assembly comprises a retainer and a delivery member hub configured to be attached to a medicament delivery member, wherein the retainer comprises a body attached to the housing and a central cavity extending through the body in the direction of the longitudinal axis, wherein the central cavity is configured to be lined up with a proximal end of the second medicament container when the second medicament container is accommodated in the adapter body, and wherein the delivery member hub is movable within the central cavity from a proximal position where the delivery member hub is configured to keep the medicament delivery member spaced apart from the second medicament container to a distal position where the delivery member hub is configured to enable the medicament delivery member to establish fluid communication with the second medicament container.

17. The sub-assembly according to claim 16, wherein the retainer comprises a delivery member fastener extending from a surface of the central cavity of the body of the retainer, wherein the delivery member hub comprises a counter delivery member fastener extending from an outer surface of the delivery member hub, wherein the counter delivery member fastener is engaged with the delivery member fastener, wherein one of the delivery member fastener of the retainer and the counter delivery member fastener of the delivery member hub is a screw thread, and wherein the other one of the delivery member fastener of the retainer and the counter delivery member fastener of the delivery member hub is a thread follower.

18. The sub-assembly according to claim 17, wherein the thread follower is a screw thread structure, a protruding pin, or a helical protrusion.

19. The sub-assembly according to claim 16, wherein the body of the retainer comprises a distally directed surface configured to be in contact with a shoulder section of the second medicament container.

20. The sub-assembly according to claim 16, wherein the body of the retainer comprises a distally extending section, wherein the body of the retainer comprises a housing fastener configured to connect to the housing so that the retainer is attached to the housing, and wherein the housing fastener of the retainer is a part of the distally extending section of the body of the retainer.

21. The sub-assembly according to claim 20, wherein the distally extending section of the body of the retainer is a distally extending arm.

22. The sub-assembly according to claim 20, wherein the adapter body is at least partially located between the distally extending section of the body of the retainer and the longitudinal axis in a direction transverse to the longitudinal axis, and wherein the housing fastener of the retainer is engaged with a part of the housing so that the retainer is directly attached to the housing.

23. The sub-assembly according to claim 20, wherein the adapter body is at least partially located between the distally extending section of the body of the retainer and the housing in a direction transverse to the longitudinal axis, wherein the adapter body is engaged with the housing, and wherein the housing fastener of the retainer is engaged with the adapter body so that the retainer is indirectly attached to the housing via the adapter body.

24. The sub-assembly according to claim 20, wherein the housing fastener comprises a snap-fit ledge extending in a direction transverse to the longitudinal axis.

25. The sub-assembly according to claim 16, wherein the adapter comprises an extending section extending from the distal end of the adapter body in a direction transverse to the longitudinal axis and towards the longitudinal axis, and wherein the extending section comprises a proximally directed surface configured to engage with a distal end of the second medicament container so that the second medicament container is axially immovable within the housing.

26. The sub-assembly according to claim 16, wherein the adapter body comprises a sleeve configured to surround the second medicament container.

27. The sub-assembly according to claim 26, wherein the sleeve is formed by a distal sleeve and a proximal sleeve attached to the distal sleeve or wherein the sleeve is formed by two halves that are each a semi-cylindrical sleeve body.

28. A method of assembling a sub-assembly of a cassette of a medicament delivery device, the method comprising the steps of:
providing a group of housings, wherein the housings in the group of housings are identical;
providing a group of medicament containers, wherein the group of medicament containers comprises a first medicament container with a first dimension and a first volume and a second medicament container with a second dimension, and wherein the second dimension is different from the first dimension and capable of containing no more than the first volume of a medicament;
providing a group of delivery member holder assemblies, wherein each of the delivery member holder assemblies in the group of delivery member holder assemblies comprises a retainer and a delivery member hub configured to be attached to a medicament delivery member, wherein the retainer comprises a body and a central cavity extending through the body in the direction of the longitudinal axis, wherein the delivery member hub is movable within the central cavity;
providing an adapter;
selecting a medicament container from the group of medicament containers;
selecting a delivery member holder assembly from the group of the delivery member holder assemblies;
inserting the selected medicament container into the housing when the first medicament container is selected or connecting the selected medicament container to the adapter when the second medicament container is selected, and inserting the selected medicament container with the adapter into the housing; and
attaching the selected delivery member holder assembly to the housing so that the central cavity of the retainer is lined up with the selected medicament container.

29. The method according to claim 28, wherein the step of providing a group of delivery member holder assemblies comprises the steps of:
providing the group of delivery member holder assemblies, wherein the group of delivery member holder assemblies comprises a first retainer configured to snugly fit to the first medicament container and a second retainer configured to snugly fit to the second medicament container; and
when the second medicament container is selected, attaching the adapter to an outer surface of the second retainer when the second retainer is selected, or attaching the adapter to an inner surface of the housing when the second retainer is selected thereby connecting the second medicament container indirectly to the adapter via the second retainer.

30. The method according to claim 28, wherein the step of providing a group of delivery member holder assemblies comprises the steps of:
providing the group of delivery member holder assemblies, wherein the delivery member holder assemblies of the group of delivery member holder assemblies are identical; and
when the second medicament container is selected, attaching the adapter to the second medicament container, thereby positioning the adapter and the second medicament container at least partially within the retainer of the delivery member holder assembly when attaching the selected delivery member holder assembly to the housing.
